(19) United States
(12) Patent Application Publication
(10) Pub. No.: US 2023/0062453 A1
(43) Pub. Date: Mar. 2, 2023

(54) SHOE INSOLE APPARATUS CAPABLE OF BUFFERING AND MASSAGING

(71) Applicant: Lang Suc SU, Hwaseong-si, Gyeonggi-do (KR)

(72) Inventor: Lang Suc SU, Hwaseong-si, Gyeonggi-do (KR)

(21) Appl. No.: 17/800,224

(22) PCT Filed: Mar. 5, 2020
(86) PCT No.: PCT/KR2020/003091

§ 371 Date: Aug. 17, 2022

(30) Foreign Application Priority Data
Feb. 19, 2020 (KR) 10-2020-0020291

(51) Int. Cl. A43B 17/16

(52) U.S. Cl. CPC A43B 17/16

(57) ABSTRACT

A shoe insole apparatus capable of buffering and massaging includes a pad part laid on a bottom surface inside a shoe and a buffering part which adheres to a portion including a central portion to a heel portion of the pad part and absorbs shock applied to a heel of a wearer, wherein the buffering part includes a body part of which a height decreases from the heel portion of the pad part toward a ball portion, a buffering groove which is positioned in an upper portion of the heel portion of the pad part, passes through the body part, and is formed in a quadrangular shape, and a plurality of buffering plates which are formed to extend to connect two inner surfaces facing each other among inner surfaces of the buffering groove and are in contact with the heel of the wearer.

### TECHNICAL FIELD

The present invention relates a shoe insole apparatus capable of buffering and massaging, and more specifically, to a shoe insole apparatus capable of buffering and massaging, which buffers shock applied to a heel of a wearer and massages the foot of the wearer through acupressure.

### BACKGROUND ART

The feet are generally referred to as the second heart, and the importance of the feet is greatly highlighted (recognized).

A foot is divided into a forefoot mainly made up of the toes, a midfoot positioned in a middle portion other than the toe portion and the heel portion, and a hindfoot mainly made up of the heel.

The forefoot is made up of one big toe and the other four toes and receives a force when moving forward, the midfoot is made up of metatarsal bones, a front portion of the midfoot is connected to the forefoot, a rear portion of the midfoot is connected to the hindfoot and serves to support the body (weight), and the hindfoot is mainly made up of heel bones, absorbs an initial shock of the weight when walking, and serves to support the weight and balance when standing.

A generally used shoe (running shoe or the like) is mainly divided into an upper part which protects the entire foot including the top of the foot, an outsole including a midsole and a bottom sole which support and protect the sole of the foot and buffer shock, and an insole which is installed in the outsole, actually supports and protects the sole of the foot, and buffers the shock.

Generally, custom shoes manufactured to be fitted to a wearer's feet and ready-made shoes (ready-made running shoes or the like) manufactured based on standard dimensions are used as shoes (running shoes or the like), and in modern life, ready-made shoes are mainly used (worn).

Ready-made shoes are standardized. Particularly, the insoles are also standardized (formed in the same shape). However, even when feet of individuals have the same dimensions, curved shapes and forms (three-dimensional forms) of the feet (toes, soles, and hindfeet) are completely different.

In spite of that, each individual is required to wear the standardized ready-made shoe, and the insole of the standardized ready-made shoe does not fit the characteristics of each individual's foot, causing discomfort in wear and unstable posture, thus increasing foot fatigue.

Recently, even when ready-made shoes are purchased, custom insoles fit to the wearer's feet (in a curved shape and three-dimensional form) are also worn.

A conventional custom insole is molded using a method in which an oven (thermoforming apparatus) including a heating device is separately manufactured, an insole is put in the oven (thermoforming apparatus) and preheated, the insole preheated by the oven (thermoforming apparatus) is put in a shoe, and the shoe is worn to mold the custom insole.

Another conventional insole is molded using a method in which a foot molding container filled with a foot frame molding liquid is heated using a microwave or hot water to preheat the foot frame molding liquid, a foot is put on the preheated foot frame molding liquid to mold a sole, a sole pattern molding liquid is poured on a portion in which the sole is molded to mold a sole pattern, and the separated insole pattern is pressed against a thermoforming injection molding part (insole) to form the insole.

Still another conventional insole is molded using a method in which a custom insole is preheated using an electric oven, the preheated custom insole is loaded on a mold, and a user stands on the preheated custom insole before the preheated custom insole hardens and waits until the preheated custom insole hardens to mold the custom insole.

However, when insoles are preheated to mold custom insoles using the above-described apparatuses and methods, a phenomenon in which a front half portion rolls rearward due to a thickness and a material of the insole occurs, a phenomenon in which a midportion inflates occurs, and a phenomenon in which a custom insole cannot be easily molded due to the deformation occurs.

In addition, since the molding is performed (by pressing using a foot or imprinting a foot) in a state in which the preheated insole is completely exposed to the outside, a phenomenon in which heat of the insole easily dissipates and the molding of the custom insole is not easily performed occurs.

Particularly, since the conventional insoles molded as described above cannot supply air, cool soles of the feet cannot be maintained, and sweat is generated, thereby breeding bacteria and causing the feet to smell very bad.

In addition, since the conventional insoles are formed of soft materials in order to absorb shock applied to feet of wearers, a massage function of acupressure on the foot of the wearer using a material with a certain rigidity or higher cannot be added.

### Technical Problem

The present invention is directed to providing a shoe insole apparatus capable of buffering and massaging which buffers shock applied to a heel of a wearer and massages the foot of the wearer through acupressure.

Objectives of the present invention are not limited to the above-described objectives, and other objectives and advantages of the present invention may be understood from the following descriptions and clearly understood from embodiments of the present invention. In addition, it may be seen that the objectives and the advantages of the present invention may be realized using elements described in the appended claims and combinations thereof.

### Technical Solution

One aspect of the present invention provides a shoe insole apparatus capable of buffering and massaging, the shoe insole apparatus including a pad part laid on a bottom surface inside a shoe and a buffering part which adheres to a portion including a central portion to a heel portion of the pad part and absorbs shock applied to a heel of a wearer, wherein the buffering part includes a body part of which a height decreases from the heel portion of the pad part toward a ball portion, a buffering groove which is positioned in an upper portion of the heel portion of the pad part, passes through the body part, and is formed in a quadrangular shape, and a plurality of buffering plates which are formed to extend to connect two inner surfaces facing each other among inner surfaces of the buffering groove and are in contact with the heel of the wearer.

In the buffering groove, a vertical length along a vertical axis parallel to a longitudinal direction of the shoe and a horizontal length along a horizontal axis perpendicular to the vertical axis may be proportional to a length of the shoe.

The plurality of buffering plates may include protruding buffering parts which are formed to protrude in a longitudinal direction of the shoe to increase an area of the buffering plates.

The shoe insole apparatus capable of buffering and massaging may further include a sensing part configured to measure a first separation distance between an inner surface on which the plurality of buffering plates are not formed among inner surfaces of the buffering groove and any one buffering plate of the plurality of buffering plates and a processor which determines whether a lifetime of the buffering part has ended on the basis of whether the first separation distance is less than or equal to a preset reference separation distance, and when the first separation distance is less than or equal to the preset reference separation distance, determines that the lifetime of the buffering part has ended.

The sensing part may measure a second separation distance between a first shoe and a second shoe of a pair of shoes, first movement information of the first shoe, and second movement information of the second shoe using signal strength information.

The processor may determine whether the wearer is walking on the basis of the first movement information and the second movement information, when the wearer is determined to be walking according, calculate a separation distance average of second separation distances, and determine whether the walking of the wearer is unbalanced on the basis of the separation distance average.

### Advantageous Effects

According to the present invention, a wearer's fatigue due to walking or running can be reduced by buffering shock applied to a heel of the wearer.

In addition, blood circulation of the wearer can be promoted by massaging the foot of the wearer through acupressure.

### DESCRIPTION OF DRAWINGS

FIG. 1 is a view illustrating a shoe insole apparatus capable of buffering and massaging according to various embodiments of the present invention.

FIG. 2 is a block diagram illustrating components of a shoe insole apparatus capable of buffering and massaging according to one embodiment of the present invention.

FIG. 3 is a perspective view illustrating a buffering part of the shoe insole apparatus capable of buffering and massaging according to one embodiment of the present invention.

FIG. 4 is a cross-sectional view illustrating the buffering part of the shoe insole apparatus capable of buffering and massaging according to one embodiment of the present invention.

FIG. 5 is a block diagram illustrating components of a shoe insole apparatus capable of buffering and massaging according to another embodiment of the present invention.

FIG. 6 is a perspective view illustrating a buffering part of the shoe insole apparatus capable of buffering and massaging according to another embodiment of the present invention.

FIG. 7 is a cross-sectional view illustrating the buffering part of the shoe insole apparatus capable of buffering and massaging according to another embodiment of the present invention.

### BEST MODES OF THE INVENTION

A shoe insole apparatus capable of buffering and massaging in order to achieve the technical objectives may include a pad part laid on a bottom surface inside a shoe and a buffering part which adheres to a portion including a central portion to a heel portion of the pad part and absorbs shock applied to a heel of a wearer, wherein the buffering part includes a body part of which a height decreases from the heel portion of the pad part toward a ball portion, a buffering groove which is positioned in an upper portion of the heel portion of the pad part, passes through the body part, and is formed in a quadrangular shape, and a plurality of buffering plates which are formed to extend to connect two inner surfaces facing each other among inner surfaces of the buffering groove and are in contact with the heel of the wearer.

### Modes of the Invention

Hereinafter, various embodiments of the present invention will be described with reference to the accompanying drawings. However, this is not intended to limit the present invention to the specific embodiments, and it should be appreciated that modifications, equivalents, and/or alternatives of the embodiments of the present invention are encompassed in the present invention. In relation to the descriptions of the drawings, the same symbol may be assigned to similar components.

In the present specification, the expression “comprise,” “may comprise,” “includes,” “may include,” or the like indicates the presence of the corresponding features (for example, numerical values, functions, operations, or components of parts or the like) and does not preclude the presence of additional features.

In present specification, the phrase, “A or B,” “at least one of A or/and B,” “one or more of A or/and B,” or the like may include all possible combinations among items thus listed. For example, “A or B,” “at least one of A and B,” or “at least one of A or B” may refer to all cases of “(1) including at least one A,” (2) “including at least one B” or “(3) including both at least one A and at least one B.”

The term “first,” “second,” or the like used in the present specification may describe various components regardless of order and/or importance, is used to distinguish one component from another component, and does not limit the component. For example, a first wearer apparatus and a second wearer apparatus may indicate different wearer apparatuses regardless of order or importance. For example, a first component may be referred to as a second component, and similarly, the second component may also be reversely referred to as the first component without departing from the scope defined in the present specification.

It should be understood that, when a certain element (for example, a first element) is referred to as “being (operatively or communicatively) connected to” or “coupled with/to” to another element (for example, a second element), the certain element may be directly connected to the other element or connected through another element (for example, a third element). In contrast, it may be understood that, when a certain element (for example, a first element) is referred to as “being directly connected to” or “directly coupled with/to” another element (for example, a second element), there is no element (for example, a third element) between the certain element and the other element.

The expression “configured (or set) to” used in the present specification may be used interchangeably with, for example, “suitable for,” “having the capacity to,” “designed to,” “adapted to,” “made to,” or “capable of” according to a situation. The expression “configured (or set) to” does not necessarily mean that hardware is “specifically designed to” do something. Instead, in some situations, the expression “an apparatus configured to” may mean that the apparatus “may be configured to” perform an action along with other apparatuses or components. For example, the expression “a processor configured (or set) to perform A, B, and C” may refer to a designated processor (for example, an embedded processor) for performing the corresponding operations or a generic-purpose processor (for example, a central processing unit (CPU) or application processor) capable of performing the corresponding operations by executing one or more software programs stored in a memory.

The terminology used herein is for the purpose of describing particular embodiments only and is not intended to limit a range of any other embodiments. The singular forms are intended to include the plural forms, unless the context clearly indicates otherwise. Terms including technical and scientific terms used herein have meanings which are the same as meanings generally understood by those skilled in the art. Terms, such as those defined in commonly used dictionaries, may be interpreted as having meanings that are consistent with or similar to their meanings in the context of the relevant art and will not be interpreted in an idealized or overly formal sense unless expressly so defined here. In some cases, even terms defined in the present specification should not be interpreted to exclude embodiments of the present specification.

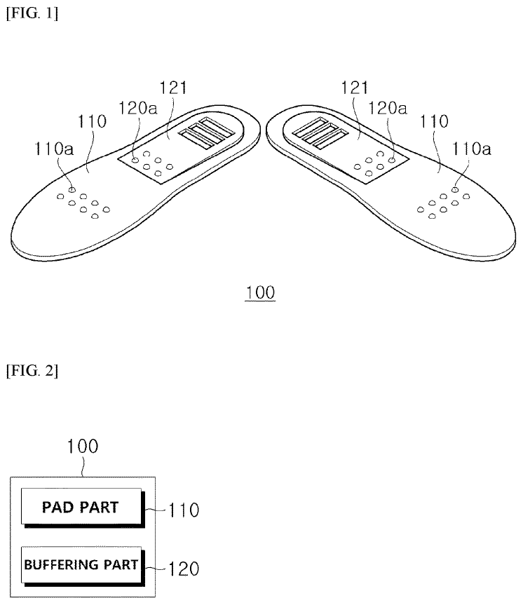

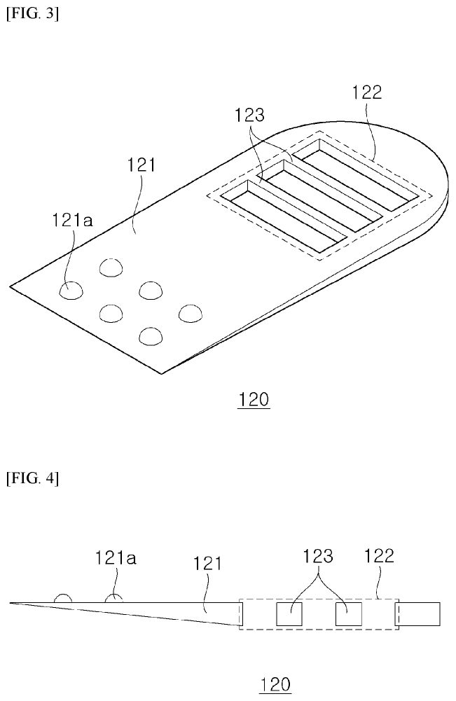

FIG. 1 is a view illustrating a shoe insole apparatus 100 capable of buffering and massaging according to various embodiments of the present invention, and FIG. 2 is a block diagram illustrating components of the shoe insole apparatus 100 capable of buffering and massaging according to one embodiment of the present invention. FIG. 3 is a perspective view illustrating a buffering part of the shoe insole apparatus 100 capable of buffering and massaging according to one embodiment of the present invention, and FIG. 4 is a cross-sectional view illustrating the buffering part of the shoe insole apparatus 100 capable of buffering and massaging according to one embodiment of the present invention.

Referring to FIGS. 1 to 4, the shoe insole apparatus 100 capable of buffering and massaging may include a pad part 110 and a buffering part 120.

Specifically, the pad part 110 may be formed in a pad shape having a predetermined thickness to be laid on a bottom surface inside a shoe.

In addition, the pad part 110 may be formed in the shape corresponding to a shape of the bottom surface inside the shoe to cover the entire bottom surface inside the shoe.

The pad part 110 may be formed of polyurethane with elasticity.

The buffering part 120 may adhere to a portion including a central portion to a heel portion of the pad part 110 and absorb shock applied to a heel of a wearer.

Meanwhile, the pad part 110 may include a plurality of first massage protrusions 110a formed to protrude upward at positions of acupoints corresponding to acupoints of the foot of the wearer.

The plurality of first massage protrusions 110a may be formed in a hemisphere shape corresponding to a predetermined curvature and formed of a material with predetermined rigidity.

When the wearer wears the shoe and walks or runs, the weight of the wearer is applied to the shoe, and the plurality of first massage protrusions 110a can promote blood circulation of the wearer by pressing the acupoints of the foot of the wearer due to the weight of the wearer.

Accordingly, the plurality of first massage protrusions 110a can achieve a massage effect of acupressure on the foot of the wearer while the wearer wears the shoe.

The buffering part 120 may be formed of polyurethane with elasticity.

Specifically, the buffering part 120 may include a body part 121, a buffering groove 122, and a plurality of buffering plates 123.

The body part 121 may adhere to a portion including the central portion to the heel portion of the pad part 110 and be formed so that a height decreases from the heel portion of the pad part toward a ball portion.

In this case, in the pad part 110, the heel portion may be a region in contact with the heel of the wearer, the ball portion may be a region in contact with the pad part 110 when the wearer lifts the heel, and the central portion may be a region between the ball portion and the heel portion.

Meanwhile, the body part 121 may include a plurality of second massage protrusions 121a formed to protrude upward from an upper surface at positions of acupoints corresponding to acupoints of the foot of the wearer.

The plurality of second massage protrusions 121a may be formed in a hemisphere shape corresponding to a predetermined curvature and formed of a material with predetermined rigidity.

When the wearer wears the shoe and walks or runs, the weight of the wearer is applied to the shoe, and the plurality of second massage protrusions 121a can promote blood circulation of the wearer by pressing the acupoints of the foot of the wearer due to the weight of the wearer.

Accordingly, the plurality of second massage protrusions 121a can achieve a massage effect of acupressure on the foot of the wearer while the wearer wears the shoe.

The buffering groove 122 may be positioned in an upper portion of the heel portion of the pad part 110, may pass through the body part 121, and may be formed in a quadrangular shape.

In the buffering groove 122, a vertical length y along a vertical axis parallel to a longitudinal direction of the shoe and a horizontal length x along a horizontal axis perpendicular to the vertical axis may be proportional to a length of the shoe.

That is, the buffering groove 122 is formed so that the vertical length y along the vertical axis and the horizontal length x along the horizontal axis become long as the length of the shoe in which the shoe insole apparatus 100 capable of buffering and massaging is installed is long, and the vertical length y along the vertical axis and the horizontal length x along the horizontal axis becomes short as the length of the shoe is short.

The plurality of buffering plates 123 may be formed to extend to connect two inner surfaces facing each other among inner surfaces of the buffering groove 122 and be in contact with the heel of the wearer.

The plurality of buffering plates 123 may be formed of a material with elasticity and contracted by the heel of the wearer, thereby buffering shock applied to the heel of the wearer.

In addition, the plurality of buffering plates 123 may be disposed apart from each other in the buffering groove 122, and when an external force is applied to the plurality of buffering plates 123 from the heel of the wearer, gaps between the plurality of buffering plates 123 may widen or narrow to buffer shock applied to the heel of the wearer.

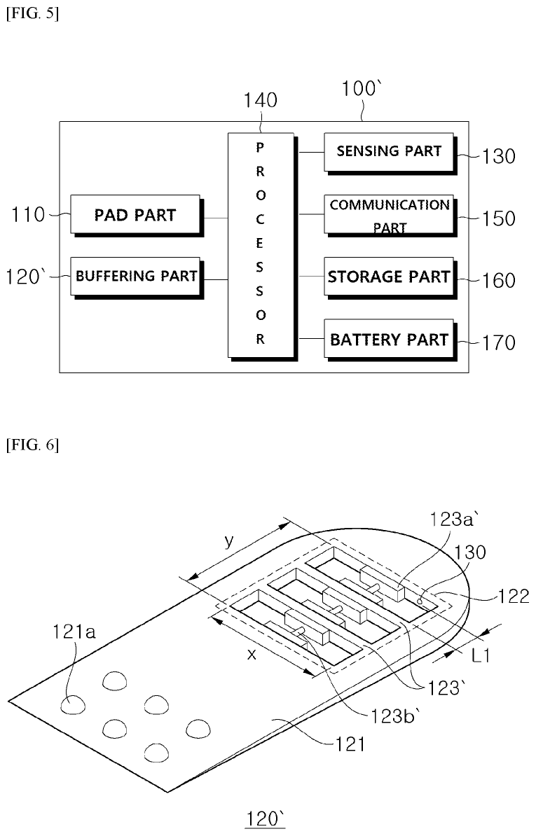

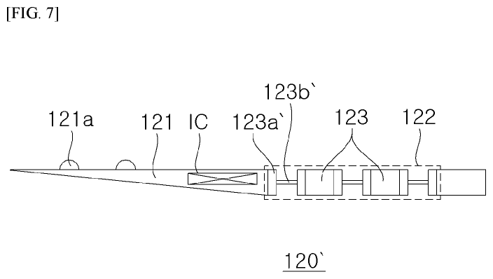

FIG. 5 is a block diagram illustrating components of a shoe insole apparatus 100′ capable of buffering and massaging according to another embodiment of the present invention, and FIG. 6 is a perspective view illustrating a buffering part 120′ of the shoe insole apparatus 100′ capable of buffering and massaging according to another embodiment of the present invention. FIG. 7 is a cross-sectional view illustrating the buffering part 120′ of the shoe insole apparatus 100′ capable of buffering and massaging according to another embodiment of the present invention.

In the shoe insole apparatus 100′ capable of buffering and massaging according to another embodiment of the present invention, a plurality of buffering plates 123′ may further include protruding buffering parts 123a′ and coupling shafts 123b′ when compared to the shoe insole apparatus 100 capable of buffering and massaging according to one embodiment of the present invention. The shoe insole apparatus 100′ capable of buffering and massaging may further include a sensing part 130, a processor 140, a communication part 150, a storage part 160, and a battery part 170.

Accordingly, repeated description will be omitted.

The shoe insole apparatus 100′ capable of buffering and massaging according to another embodiment of the present invention may include a pad part 110, a buffering part 120′, the sensing part 130, the processor 140, the communication part 150, the storage part 160, and the battery part 170.

Each of the plurality of buffering plates 123′ included in the buffering part 120′ may include the protruding buffering part 123a′ formed to protrude in a longitudinal direction of a shoe to increase an area of the buffering plate 123′.

The protruding buffering part 123a′ is formed to protrude from a central portion of the buffering plate 123′ in the longitudinal direction to increase an area to which an external force is applied from a heel of a wearer.

Meanwhile, each of the plurality of buffering plates 123′ may include a coupling shaft 123b′ which is disposed between the protruding buffering parts 123a′ and couples the protruding buffering parts 123a′.

The coupling shaft 123b′ may be formed to extend from any one protruding buffering part 123a′ to be connected to the protruding buffering part 123a′ which is most adjacent to any one protruding buffering part 123a′. Accordingly, even when an excessive external force is applied to the plurality of buffering plates 123′ from the heel of the wearer, a phenomenon in which a separation distance between the plurality of buffering plates 123′ becomes greater than a reference distance and thus shock applied to the heel of the wearer may not be buffered can be prevented.

Meanwhile, the sensing part 130 may measure a first separation distance L1 between an inner surface on which the plurality of buffering plates 123′ are not formed among inner surfaces of a buffering groove 121 and any one buffering plate 123′ among the plurality of buffering plates 123′.

To this end, the sensing part 130 may include a distance measuring sensor which is disposed on the inner surface on which the plurality of buffering plates 123′ are not formed among the inner surfaces of the buffering groove 121 and measures a distance using one or more among infrared rays or ultrasonic waves.

In addition, the sensing part 130 may measure a second separation distance between a first shoe and a second shoe of a pair of shoes, first movement information of the first shoe, and second movement information of the second shoe using signal strength information.

To this end, the sensing part 130 may include a Bluetooth communication module which is disposed in each of the first shoe and the second shoe, transmits and receives a signal through Bluetooth communication to measure the signal strength information, and outputs the second separation distance on the basis of the signal strength information.

In addition, the sensing part 130 may include an acceleration sensor which is disposed in each of the first shoe and the second shoe and measures the first movement information and the second movement information

The processor 140 may control operations of the sensing part 130, the communication part 150, the storage part 160, and the battery part 170.

The processor 140 may include one or more cores (not shown), a graphic processor (not shown), and/or a connecting path (for example, a bus) through which a signal is transmitted and received between different components.

The processor 140 according to one embodiment may be configured to perform an operation of the shoe insole apparatus 100 capable of buffering and massaging by executing one or more instructions stored in the storage part 160.

Specifically, the processor 140 may determine whether a lifetime of the buffering part 120′ has ended on the basis of whether the first separation distance is smaller than or equal to a preset reference separation distance, and when the first separation distance is smaller than or equal to the preset reference separation distance, the processor 140 may determine that the lifetime of the buffering part 120′ has ended.

In this case, the preset reference separation distance may be the first separation distance when a state in which elasticity of the plurality of buffering plates 123′ is lost and the plurality of buffering plates 123′ are widened toward the inner surfaces of the buffering groove 121 is not restored.

Then, when the processor 140 determines that the lifetime of the buffering part 120′ has ended, the processor 140 may control the communication part 150 to transmit a lifetime end notification signal to notify of the end of the lifetime of the buffering part 120′ to the outside.

In this case, the outside may be a wearer's terminal used by the wearer.

Meanwhile, the processor 140 may determine whether the wearer is walking on the basis of the first movement information and the second movement information, and when the wearer is determined to be walking, the processor 140 may calculates a separation distance average of second separation distances and determine whether the walking of the wearer is unbalanced on the basis of the separation distance average.

Specifically, when an acceleration average of first acceleration information from the first movement information and an acceleration average of second acceleration information from the second movement information are included within a preset reference acceleration range, the processor 140 may determine that the wearer is walking.

In this case, the preset reference acceleration range may be an acceleration average measured when the wearer is walking.

Then, when the wearer is walking, the processor 140 may calculate a first separation distance average of the second separation distance for a first period in which the first movement information indicates no movement and a second separation distance average of the second separation distance for a second period in which the second movement information indicates no movement.

That is, in a situation in which the wearer is walking, the processor 140 may calculate the first separation distance average when, in a state in which any one foot stops, the other foot moves, and the second separation distance average when, in a state in which the other foot stops, the one foot moves.

Finally, when an average difference between the first separation distance average and the second separation distance average is greater than or equal to a preset reference average difference, the processor 140 may determine that the walking of the wearer is unbalanced.

To this end, the sensing part 130 may transmit a result measured from each of the first shoe and the second shoe to the processor 140 through short distance wireless communication.

In addition, the first communication part 150 may be connected to a communication network to communicate with the outside using general-purpose communication.

To this end, the communication part 150 may include a general-purpose communication module configured to perform general-purpose communication. In this case, the general-purpose communication may be communication using an Internet network, or may use at least one cellular communication protocol among, for example, Long-Term Evolution (LTE), LTE Advanced (LTE-A), Code Division Multiple Access (CDMA), Wideband CDMA (WCDMA), Universal Mobile Telecommunications System (UMTS), wireless broadband (WiBro), and Global System for Mobile Communications (GSM).

The storage part 160 may store programs (one or more instructions) for processing or controlling of the processor 140. The programs stored in the storage part 160 may be divided into a plurality of modules.

The battery part 170 may be disposed in each of the first shoe and the second shoe and may supply power to components of the shoe insole apparatus 100 capable of buffering and massaging.

Meanwhile, the sensing part 130, the processor 140, the communication part 150, the storage part 160, and the battery part 170 may be formed as one integrated circuit (IC) and inserted into the buffering part 120′.

The present invention has been particularly described above with reference to exemplary embodiments. It will be understood by those skilled in the art that the present invention may be implemented in modified forms within a range not departing from the essential spirit of the present invention. Therefore, the embodiments should be considered in a descriptive sense only and not for purposes of limitation. Therefore, the scope of the present invention is defined not by the above description but by the appended claims, and all differences within the equivalents of the scope will be construed as being included in the present invention.

Although the present invention has been described above with reference to the limited specific embodiments and drawings, the present invention is not limited thereto, and various modifications and changes may be made by those skilled in the art in the technical spirit of the present invention and equivalents of the scope which will be defined in the claims below.

1. A shoe insole apparatus capable of buffering and massaging, the shoe insole apparatus comprising:
a pad part laid on a bottom surface inside a shoe; and
a buffering part which adheres to a portion including a central portion to a heel portion of the pad part and absorbs shock applied to a heel of a wearer,
wherein the buffering part includes a body part of which a height decreases from the heel portion of the pad part toward a ball portion,
a buffering groove which is positioned in an upper portion of the heel portion of the pad part, passes through the body part, and is formed in a quadrangular shape, and
a plurality of buffering plates which are formed to extend to connect two inner surfaces facing each other among inner surfaces of the buffering groove and are in contact with the heel of the wearer.

2. The shoe insole apparatus of claim 1, wherein, in the buffering groove, a vertical length along a vertical axis parallel to a longitudinal direction of the shoe and a horizontal length along a horizontal axis perpendicular to the vertical axis are proportional to a length of the shoe.

3. The shoe insole apparatus of claim 2, wherein the plurality of buffering plates include protruding buffering parts which are formed to protrude in a longitudinal direction of the shoe to increase an area of the buffering plates.

4. The shoe insole apparatus of claim 1, further comprising:
a sensing part configured to measure a first separation distance between an inner surface on which the plurality of buffering plates are not formed among inner surfaces of the buffering groove and any one buffering plate of the plurality of buffering plates; and
a processor which determines whether a lifetime of the buffering part has ended on the basis of whether the first separation distance is less than or equal to a preset reference separation distance, and when the first separation distance is less than or equal to the preset reference separation distance, determines that the lifetime of the buffering part has ended.

5. The shoe insole apparatus of claim 1, wherein:
the sensing part measures a second separation distance between a first shoe and a second shoe of a pair of shoes, first movement information of the first shoe, and second movement information of the second shoe using signal strength information; and
the processor determines whether the wearer is walking on the basis of the first movement information and the second movement information, and when the wearer is determined to be walking, calculates a separation distance average of second separation distances, and determines whether the walking of the wearer is unbalanced on the basis of the separation distance average.
